(19) United States
(12) Patent Application Publication
(10) Pub. No.: US 2019/0314546 A1
(43) Pub. Date: Oct. 17, 2019

(54) ANTIBIOTIC POLYMETHYLMETHACRYLATE BONE CEMENT

(71) Applicant: HERAEUS MEDICAL GMBH, WEHRHEIM (DE)

(72) Inventors: SEBASTIAN VOGT, ERFURT (DE); SUSANN KLIMAS, BAD HOMBURG v.d.H. (DE)

(73) Assignee: HERAEUS MEDICAL GMBH, WEHRHEIM (DE)

(21) Appl. No.: 16/432,333

(22) Filed: Jun. 5, 2019

Related U.S. Application Data
(62) Division of application No. 15/386,522, filed on Dec. 21, 2016, now abandoned.

(30) Foreign Application Priority Data
Dec. 22, 2015 (DE) 10 2015 226 501.3

(51) Int. Cl. A61L 24/06; A61L 24/00; A61L 27/54; A61L 27/16; A61L 27/50

(52) U.S. Cl. CPC A61L 24/06; A61L 24/0015; A61L 27/54; A61L 2300/406; A61L 24/001; A61L 27/50; A61L 2430/02; A61L 27/16

(57) ABSTRACT

An antibiotic polymethylmethacrylate bone cement that is composed of methylmethacrylate, at least one polymethylmethacrylate or a polymethylmethacrylate-copolymer, at least one polymerisation initiator, such as in radical initiator, at least one polymerisation accelerator, and at least one radiopaquer, whereby the components are present in a powdered component and a liquid monomer component or in two components that are pasty at room temperature. The polymethylmethacrylate bone cement comprises the cyclical lipopeptide, daptomycin, a pharmacologically tolerable salt of daptomycin, a solvate and/or a hydrate containing daptomycin and at least one calcium salt, which preferably comprises at least two different release profiles. The bone cement is useful in one-stage or two-stage septic revision surgery.

The present application is a division of U.S. patent application Ser. No. 15/386,522, filed Dec. 21, 2016, now pending, which, in turn, claims priority of German Patent Application No. 10 2015 226 501.3, filed Dec. 22, 2015, the entire contents of which are incorporated herein by reference.

The object of the invention is an antibiotic polymethylmethacrylate bone cement that comprises methylmethacrylate, at least one polymethylmethacrylate or a polymethylmethacrylate-copolymer, at least one polymerisation initiator, at least one polymerisation accelerator, and at least one radiopaquer, whereby the components can be present in a powdered component and a liquid monomer component or in two components that are pasty at room temperature. The polymethylmethacrylate bone cement according to the invention comprises the cyclical lipopeptide, daptomycin, a pharmacologically tolerable salt of daptomycin, a solvate and/or a hydrate containing daptomycin and at least one calcium salt, which preferably comprises at least two different release profiles.

The subject matter of the invention is an antibiotic bone cement intended for anchoring of revision articular endoprostheses in the scope of one-stage and two-stage septic revision surgeries, in which gram -positive bacteria, in particular methicillin-resistant staphylococci (MRSA, MRSE) or vancomycin-resistant staphylococci are the cause of the underlying infection. In addition, the antibiotic polymethylmethacrylate bone cement is also well-suited for the production of spacers as temporary place-holders in two-stage septic revision surgeries.

Articular endoprostheses are used extensively and very successfully in a broad range of articular diseases aiming to maintain the mobility of the patients. Unfortunately, a small fraction of the patients suffers infections at the articular endoprostheses and in the surrounding bone tissue and soft tissue. To treat these infections, it is very common to perform a one-stage or two-stage revision of the articular endoprosthesis. Revision polymethylmethacrylate bone cements containing an antibiotic or two or more antibiotics have proven expedient for permanent mechanical fixation of the revision articular endoprostheses. Said antibiotics protect the revision articular endoprosthesis and the surrounding bone tissue and soft tissue, at least right after the surgery, from renewed microbial colonisation. Aside from the individualised admixture of antibiotics by the physician, industrially produced revision polymethylmethacrylate bone cements have proven expedient. Accordingly, Heraeus Medical GmbH manufactures and distributes the revision polymethylmethacrylate bone cements, CORAL® G+C and CORAL® G+V. CORAL® G+C contains the combination of gentamicin and clindamycin. CORAL® G+V contains the combination of gentamicin and vancomycin. The combination of gentamicin and vancomycin is particularly well-suited, thus far, if the infection of the articular endoprosthesis is caused by methicillin-resistant staphylococci (MRSA, MRSE). However, vancomycin-resistant strains of staphylococci and enterococci have been known for a number of years as well. It is to be expected that these vancomycin-resistant bacteria, aside from MRSA/MRSE currently, will assume an increasing role as the causes of joint-associated infections in the near future. Therefore, it makes sense to develop a revision polymethylmethacrylate bone cement that contains at least one antibiotic that is effective against vancomycin-resistant bacteria.

Implant-associated infections by multi-resistant gram-positive bacteria, in particular by vancomycin-resistant bacteria, are extremely difficult to treat and are a serious hazard for the patients. Therefore, for these difficult implant-associated infections, revision cements are desirable that contain antibiotics expected to provide safe local protection from reinfection by these problematic germs.

Daptomycin (CAS 103060-53-3) is an antibiotic that is effective against vancomycin-resistant bacteria. Daptomycin is a cyclic lipopeptide. Its mechanism of action is quite different from that of glycopeptide antibiotics such as vancomycin and teicoplanin. As a result, it generally has an antimicrobial effect even against vancomycin-resistant gram-positive bacteria. Daptomycin becomes embedded in the cellular membrane of gram -positive bacteria and forms pores through which potassium ions from the cytoplasm of the bacterial cells can exit to reach the surroundings of the cell. Due to the efflux of potassium ions, the bacterial cells become depolarised and die. Daptomycin relies on calcium ions as cofactor for the formation of pores (G. M. Eliopoules, S. Wiley, E. Reiszner, P. G. Spitzer, G. Caputo, R. C. Moellering: In vitro and in vivo activity of LY146032, a new lipopeptide antibiotic. Antimicrob. Agents Chemother. 30 (1986) 532-535.; R. N. Jones, A. L. Barry: Antimicrobial activity and spectrum of LY146032, a lipopeptide antibiotic, including susceptibility testing recommendations. Antimicrob. Agents Chemother. 31 (1987) 625-629.).

Antibiotics that have previously been used in polymethylmethacrylate bone cements, such as gentamicin, tobramycin, vancomycin, clindamycin, erythromycin, and colistin, do not rely on cofactors for their antimicrobial efficacy. This means that the previously known antibiotics-modified polymethylmethacrylate bone cements had a local antimicrobial effect upon dissolution of the antibiotics from the cured polymethylmethacrylate bone cements by body fluids, such as wound exudate and blood, without components of the body fluids being required as cofactors for the antimicrobial efficacy.

In one-stage and two-stage septic revision surgeries, the infected bone tissue and soft tissue is subjected to radical debridement after explantation of the infected articular endoprostheses. This means that the infected bone tissue and soft tissue is removed by surgical means. Accordingly, the formation of wound exudate is observed in one-stage and two-stage revision surgeries. The purpose of the wound exudate is to transport away any debris such as cell fragments and other tissue residues. In the one-stage revision, it is formed at the boundary between the bone cement used for mechanical fixation of the revision endoprosthesis and the surface of the previously debrided bony implant bed.

In two-stage revision surgeries, wound exudate is formed firstly between the spacer surface and the bone tissue and/or soft tissue after the first debridement and then again after the second debridement during the implementation of the revision articular endoprostheses. Usually, the wound exudate is drained from the patient by means of drainages. The drainages remain indwelling in the patient until the flow of wound exudate subsides. In this context, the quantity of wound exudate can vary between individual patients from several hundred millilitres to several litres. Wound exudate consists mainly of water and proteins. It does not contain defined concentrations of alkali and alkaline earth ions. The composition varies strongly. The wound exudate dissolves antibiotics out of the surface of antibiotic-modified polymethylmethacrylate bone cements. Using antibiotics that do not require co-factors for their antimicrobial efficacy, this always guarantees antibiotic protection of the cement surface and the surrounding bone tissue by the antibiotics dissolved in the wound secretion. In contrast, the efficacy of daptomycin depends on the calcium ion concentration being sufficiently high. However, said concentration can vary individually between patients. A polymethylmethacrylate bone cement configured with daptomycin alone is therefore not assured to possess reproducible local antimicrobial efficacy.

It is the object of the invention to develop a polymethylmethacrylate bone cement with antimicrobial efficacy that contains the antibiotic daptomycin and assures a local antimicrobial efficacy of the daptomycin once the daptomycin is dissolved out of the surface of the cured cement by aqueous body fluids, such as a wound exudate and blood, regardless of the chemical composition of the surrounding aqueous body fluids.

The invention is based on finding, surprisingly, that a synchronous release of daptomycin and calcium ions from a polymethylmethacrylate bone cement over a period of several days is feasible such that a local antimicrobial efficacy of the daptomycin is evident even in the presence of body fluids that contain insufficient amounts of calcium ions. As a result, optimal local antimicrobial protection of the cement surface, of the surface of revision articular endoprostheses, and of the bone tissue surrounding the revision cement can be attained even if large quantities of wound exudate containing low to very low calcium ion contents are released by the bony implant bed and the surrounding soft tissue. The invention is based on integrating into the polymethylmethacrylate bone cement a readily water-soluble, physiologically acceptable calcium salt and a poorly water-soluble, physiologically acceptable calcium salt together with daptomycin. The readily soluble calcium salt effects a high initial release of calcium ions parallel to the high initial release of daptomycin. Subsequently, low amounts of daptomycin are released during the course of several days. This is paralleled by low amounts of calcium ions being released in delayed manner from the poorly soluble calcium salt. This ensures both the initial antimicrobial effect of daptomycin over the first two days as well as the antimicrobial effect over the subsequent days.

The object was met by the bone cements described hereinbelow as well as by a composition for use for treatment and/or prevention of a bacterial infection as also described hereinbelow as well as by a polymerisable bone cement for application in a method for treatment and/or prevention of bacterial infections described hereinbelow or by a form body or implant as also described hereinbelow. Preferred embodiments are illustrated in detail in the description.

A subject matter of the invention is an antibiotic polymerisable bone cement comprising
(i) at least one monomer for radical polymerisation;
(ii)at least one organic polymer comprising at least one polymethylmethacrylate and/or a polymethylmethacrylate copolymer, in particular the polymer can be swelled or is soluble in the monomer; and
(iii) at least one polymerisation initiator;
(iv) at least one radiopaquer;
(v) as component 1, daptomycin, a pharmacologically tolerable salt of daptomycin, a polymorphous form of daptomycin, a solvate and/or a hydrate containing daptomycin; as well as
(vi) at least one calcium salt, in particular at least one pharmacologically tolerable calcium salt.

According to the invention, the bone cement contains a rapid-release calcium salt and, optionally, a delayed-release calcium salt, preferably the at least one calcium salt that is present has a mean particle size of 1 to 250 μm. According to alternative embodiments, the at least one calcium salt that is present has at least two different mean particle sizes that are defining for the release. Accordingly, one fraction can have a mean particle size of 10 to 150 μm and the at least second fraction can have a mean particle size of 160 to 250 μm.

The mean particle sizes mentioned in the present invention all are determined by sedimentation analysis.

Moreover, preferably component 1, i.e. daptomycin, a pharmacologically tolerable salt of daptomycin, a solvate and/or a hydrate containing daptomycin has a mean particle size of 1 to 250 μm, in particular has a mean particle size of 10 μm to 250 μm, preferably of 100 to 250 μm.

The release profile of the at least one calcium salt can be controlled in different ways and manners. Accordingly, the release profile can be controlled in terms of duration concentration by means of different particle size fractions. Likewise, the release profile can be controlled by using calcium salts with different solubility in the bone cement. Another option is to incorporate the at least one calcium salt into the bone cement in the form of a formulation comprising at least one excipient.

Another subject matter of the invention is a bone cement comprising, as (vi), a combination comprising, in particular, at least two calcium salts possessing different solubility in water, preferably
a) as component 2, a water-soluble calcium salt possessing a solubility in water at room temperature of more than or equal to 5 g/l; and
b) as component 3, a poorly water-soluble calcium salt possessing a solubility in water at room temperature of less than 5 g/l; and, whereby, optionally,
c) at least one of components 1, 2 and/or 3 has a mean particle size of 1 to 250 μm.

Another subject matter of the invention is a bone cement that comprises two components A and B, whereby
(i) component A is present as a paste and comprises
(a1) at least one monomer for radical polymerisation;
(a2) at least one organic polymer comprising at least one polymethylmethacrylate and/or one polymethylmethacrylate-copolymer; and
(a3) at least one polymerisation initiator; and component B is present as a paste and comprises
(b1) at least one monomer for radical polymerisation;
(b2) at least one organic polymer comprising at least one polymethylmethacrylate and/or one polymethylmethacrylate-copolymer; and
(b3) at least one polymerisation accelerator; or
(ii) component A is present as a powder and comprises
(a1) at least one powdered polymer comprising at least one polymethylmethacrylate and/or a powdered mixture comprising polymethylmethacrylate-co-polymers;
(a2) at least one powdered radiopaquer; and
(a3) at least one polymerisation initiator; and component B is present as a liquid or paste and comprises
(b1) at least one monomer for radical polymerisation;
(b2) optionally, at least one organic polymer comprising at least one polymethylmethacrylate and/or one polymethylmethacrylate-copolymer; and
(b3) at least one polymerisation accelerator; and
whereby at least component A comprises, as
(a4), at least one of components 1, 2, and 3 selected from component 1, daptomycin, a pharmacologically tolerable salt of daptomycin, a solvate and/or a hydrate containing daptomycin, component 2, a water-soluble calcium salt possessing a solubility in water at room temperature of more than or equal to 5 g/l, and component 3, a poorly water-soluble calcium salt possessing a solubility in water at room temperature of less than 5 g/l, and/or
whereby component B is present as a paste and comprises, as
(b4), at least one of components 1, 2, and 3 selected from component 1, daptomycin, a pharmacologically tolerable salt of daptomycin, a solvate and/or a hydrate containing daptomycin, component 2, a water-soluble calcium salt possessing a solubility in water at room temperature of more than or equal to 5 g/l, and component 3, a poorly water-soluble calcium salt possessing a solubility in water at room temperature of less than 5 g/l.

Preferably, component A and/or B each independently comprise at least components 1 and 2, components 1 and 3 or components 1, 2, and 3. Alternatively, component A comprises at least component 1 and, optionally, component 2 or it comprises component 1 and, optionally, component 3 or it comprises components 1, 2, and 3, whereby concurrently component B comprises at least component 2 and, optionally, component 1 or comprises component 2 and, optionally, component 3 and, optionally, component 1 or it comprises components 1, 2, and 3. According to another alternative, component B comprises at least component 1 and, optionally, component 2 or it comprises component 1 and, optionally, component 3 or it comprises components 1, 2, and 3, whereby concurrently component A comprises at least component 2 and, optionally, component 1 or it comprises component 2 and component 3 and, optionally, component 1 or it comprises components 1, 2, and 3.

Components 1, 2, and 3 are preferably present in the polymethylmethacrylate bone cement as separate particles, whereby said particles are preferably present in the powder component or in at least one pasty component of the polymethylmethacrylate bone cement.

In an advantageous refinement of the invention, the particles of components 1, 2, and 3 are each present as a combination in a particulate formulation, whereby said particulate formulation preferably is present in the powder component or in at least one pasty component of the polymethylmethacrylate bone cement.

Another subject matter of the invention is a bone cement comprising
(v) component 1 daptomycin, a pharmacologically tolerable salt of daptomycin, a solvate and/or a hydrate containing daptomycin, and (vi) a) component 2 the water-soluble calcium salt possessing a solubility in water at room temperature of more than or equal to 5 g/l, and b) component 3 the poorly water-soluble calcium salt possessing a solubility in water at room temperature of less than 5 g/l, each independently is present in the form of particles of components 1, 2 or 3 or each independently is a particulate formulation containing, each independently, at least one of components 1, 2 and/or 3, and at least one pharmacological excipient and, optionally, each independently comprise a mean particle size of 1 to 250 μm.

Polymethylmethacrylate bone cements consisting of two pasty components and are stable upon storage and are mixed only right before the application to form a bone cement dough have been described in DE102007050762, DE102008030312, and DE102007052116. These bone cements have two cement pastes stored separately in suitable cartridges. These each contain components of a redox initiator system, aside from at least one monomer and suitable polymers. Redox initiator systems usually consist of peroxides, accelerators and, if applicable, suitable reducing agents. Radicals are formed only if all components of the redox initiator systems act in concert. For this reason, the individual components of the redox initiator system are arranged appropriately in the separate cement pastes such that these cannot trigger a radical polymerisation.

According to a variant of an embodiment, the subject matter of the invention is a bone cement, whereby at least component A comprises, as
(a4), at least one of components 1, 2, and 3, which each independently are present in the form of particles of components 1, 2 or 3 or each independently, as a particulate formulation, containing, each independently, at least one of components 1, 2 and/or 3 and, optionally, each independently comprise a mean particle size of 1 to 250 μm;
whereby component B is present as a paste and comprises, as
(b4) at least one of components 1, 2, and 3, which each independently are present in the form of particles of components 1, 2 or 3 or each independently, as a particulate formulation, containing, each independently, at least one of components 1, 2 and/or 3 and, optionally, each independently comprises a mean particle size of 1 to 250 μm;

Particularly preferably, the particles of components 1, 2, and 3 each are contained as a combination in a particulate formulation, which can be contained in component A and/or B.

According to a variant of an embodiment,
a) the bone cement is present, as (a4) and/or (b4), as particles of a combination at least of components 1 and 2 or, each independently, as a particulate formulation containing at least a combination of components 1 and 2 and at least one pharmacological excipient; or
b) the bone cement is present, as (a4) and/or (b4), as particles of a combination of components 1, 2, and 3 or, each independently, as a particulate formulation containing the combination of components 1, 2, and 3 and at least one pharmacological excipient.

Preferably, the (vi) at least one calcium salt, in particular the water-soluble calcium salt possessing a solubility in water at room temperature of more than or equal to 5 g/l is selected from at least one calcium salt of a saccharic acid, a salt of a carboxylic acid having 1 to 10 C-atoms, in particular of lactic acid, a salt of a hydroxycarboxylic acid having 1 to 10 C-atoms, a salt of a fruit acid, salt of monosaccharides, salt of disaccharides and/or the corresponding derivatives, calcium chloride, preferably CaCl₂ or a hydrate thereof.

According to particularly preferred variants of embodiments, the bone cement comprises, as
a) component 2, the water-soluble calcium salt possessing a solubility in water at room temperature of more than or equal to 5 g/l, a calcium salt comprising calcium gluconate, calcium glucuronate, calcium lactate, calcium acetate and/or calcium sorbate or a mixture containing at least two of said calcium salts.

Preferably, the (vi) at least one calcium salt, in particular the poorly water-soluble calcium salt possessing a solubility in water at room temperature of less than 5 g/l, comprises a calcium salt comprising at least one inorganic anion comprising sulfates, phosphates or salts of fatty acids having 6 to 31 C-atoms.

According to a further particularly preferred variant of an embodiment, the bone cement comprises, as b) component 3, the poorly water-soluble calcium salt possessing a solubility in water at room temperature of less than 5 g/l, comprising calciumsulfate-dihydrate, calciumsulfat-hemihydrate, alpha-tricalciumphosphate and/or beta-tricalciumphosphate.

The bone cement according to the invention can contain at least one second antibiotic in addition to daptomycin, selected from the group of the aminoglycoside antibiotics and/or the lincosamide antibiotics and/or the ansamycin antibiotics and/or the fluoroquinolone antibiotics and/or the β-lactam antibiotics. Preferably, the further antibiotic is selected from gentamicin, tobramycin, and clindamycin. The antibiotics can effect broader antibacterial action spectra by means of the antibiotic polymethylmethacrylate bone cement having a broad and antimicrobial protection range. These antibiotics attack different targets of the bacterial cells then daptomycin and thus increase the probability of the polymethylmethacrylate bone cement possessing antimicrobial efficacy.

Another subject matter of the invention is a method for producing a curable bone cement or a local release agent carrier as well as the polymerisable bone cements and the agent carrier that can be obtained according to set method by mixing components A and B. The polymerisable bone cement is formed and polymerised in order to produce the local release agent carrier.

Another subject matter of the invention is a composition, in particular as revision cement, for use in a method for the treatment and/or prevention of infections that are elicited by bacteria, in particular for the treatment and/or prevention of an infection by bacterial multi-resistant pathogens (MDR pathogens), whereby the composition comprises
(i) at least one monomer for radical polymerisation, (ii) at least one organic polymer comprising at least one polymethylmethacrylate and/or one polymethylmethacrylate-copolymer, and (iii) at least one polymerisation initiator, (iv) radiopaquer, (v) as component 1 daptomycin, a pharmacologically tolerable salt of daptomycin, a polymorphous form of daptomycin, a solvate and/or a hydrate containing daptomycin, (vi) a combination of at least two different calcium salts, whereby, in particular, one calcium salt is released rapidly and, optionally, the at least one further calcium salt is released in delayed manner, (vii) optionally, at least one polymerisation accelerator. Preferably, at least one of components 1, 2 and/or 3 has a mean particle size of 1 to 250 μm. It is preferred to consider, as calcium salts, at least one water-soluble calcium salt and/or at least one poorly water-soluble calcium salt.

The use of the bone cement or of an agent carrier for treatment and/or prevention of bacteria-elicited infections particularly preferably comprises the treatment and/or prevention of an infection by bacterial multi-resistant pathogens (MDR pathogens), such as VRSA, MRSA, VRE, etc. The following are considered to be MDR pathogens:
Methicillin-resistant Staphylococcus aureus (MRSA) strains, vancomycin-intermediary-sensitive Staphylococcus aureus (VISA) strains, vancomycin-resistant Staphylococcus aureus (DRS A) strains, extended spectrum β-lactamase (ESBL)-producing pathogens

Multiply-resistant gram-positive bacteria (MRGP/MDRGP) can comprise vancomycin/glycopeptide-resistant enterococci (VRE, GRE), penicillin-resistant pneumococci, etc. Multiply-resistant gram-negative bacteria (MRGN/MDRGN) can comprise, inter alia, Pseudomonas aeruginosa: Bacterium Acinetobacter baumannii as the cause of wound infections and sepsis.

Another subject matter of the invention is a kit for producing polymerisable bone cement, in particular as revision cement, comprising components A and B, whereby
(i) component A is present as a paste and comprises
(a1) at least one monomer for radical polymerisation;
(a2) at least one organic polymer comprising at least one polymethylmethacrylate and/or one polymethylmethacrylate-copolymer; and
(a3) at least one polymerisation initiator; and component B is present as a paste and comprises
(b1) at least one monomer for radical polymerisation;
(b2) at least one organic polymer comprising at least one polymethylmethacrylate and/or one polymethylmethacrylate-copolymer; and
(b3) at least one polymerisation accelerator; or
(ii) component A is present as a powder and comprises
(a1) at least one powdered polymer comprising at least one polymethylmethacrylate and/or a powdered mixture comprising polymethylmethacrylate-co-polymers;
(a2) at least one powdered radiopaquer; and
(a3) at least one polymerisation initiator; and component B is present as a liquid or paste and comprises
(b1) at least one monomer for radical polymerisation;
(b2) optionally, at least one organic polymer comprising at least one polymethylmethacrylate and/or one polymethylmethacrylate-copolymer; and
(b3) at least one polymerisation accelerator; and
whereby at least component A comprises, as
(a4) at least one of components 1, 2, and 3 selected from component 1, daptomycin, a pharmacologically tolerable salt of daptomycin, a solvate and/or a hydrate containing daptomycin, component 2, a water-soluble calcium salt possessing a solubility in water at room temperature of more than or equal to 5 g/l, and component 3, a poorly water-soluble calcium salt possessing a solubility in water at room temperature of less than 5 g/l, and/or, optionally,
whereby component B is present as a paste and comprises, as
(b4), at least one of components 1, 2, and 3 selected from component 1, daptomycin, a pharmacologically tolerable salt of daptomycin, a polymorphous form of daptomycin, a solvate and/or a hydrate containing daptomycin, component 2, a water-soluble calcium salt possessing a solubility in water at room temperature of more than or equal to 5 g/l, and component 3, a poorly water-soluble calcium salt possessing a solubility in water at room temperature of less than 5 g/l,
whereby the kit contains at least components 1 and 2 or at least components 1 and 3, preferably contains components 1, 2, and 3.

In this context, components 1, 2, or 3, each independently can be contained in the kit as a particulate formulation or in a combination in a particulate formulation, optionally comprising at least one pharmacological excipient.

According to a further alternative, a subject matter of the invention is a polymerised, cured bone cement for use in a method for the treatment and/or prevention of infections that are elicited by bacteria, in particular for treatment and/or prevention of an infection by bacterial multi-resistant pathogens, whereby component 1 daptomycin, a pharmacologically tolerable salt of daptomycin, a polymorphous form of daptomycin, a solvate and/or a hydrate containing daptomycin, and at least one calcium salt, preferably at least two calcium salts with different solubilities in water as components 2 and 3, in particular one calcium salt is released rapidly and, optionally, the at least one further calcium salt is released in delayed manner, whereby the release takes place in the presence of moisture, water, aqueous media, such as body fluids, or in an aqueous solution. Preferably, components 2 and 3 are selected from a component 2 a water-soluble calcium salt possessing a solubility in water at room temperature of more than or equal to 5 g/l and component 3 a poorly water-soluble calcium salt possessing a solubility in water at room temperature of less than 5 g/l.

Another subject matter of the invention is a form body obtainable by forming and polymerising the polymerisable bone cement.

Another subject matter of the invention is a surgical implant, in particular for use in a method for the treatment and/or prevention of infections elicited by bacteria, in particular by multi-resistant pathogens. Examples are a surgical implant or part of an implant, antibiotic implant, revision implant, screw, nail, surgical plate, for mechanical fixation of primary total articular endoprostheses, for mechanical fixation of revision total articular endoprostheses, for augmentation of osteoporotic bone tissue and, particularly preferably, for vertebroplasty, kyphoplasty, and augmentation of drill holes in osteoporotic bone tissue, for filling bone cavities, for femuroplasty, for the manufacture of spacers, for mechanical fixation of articular endoprostheses, for coverage of skull defects or for production of carrier materials for local antibiotics therapy or as carrier material for local release of pharmaceutically active substances.

Preferably, the (i) monomer is selected from at least one alkyl-2-acrylic acid alkylester, aryl-2-acrylic acid alkylester, arylalkyl-2-acrylic acid alkylester, each independently having 1 to 20 C-atoms in the alkyl group, each independently having 6 to 14 C-atoms in the aryl group, each independently having 6 to 14 C-atoms in the arylalkyl group, and each independently having 1 to 10 C-atoms in the alkylester group, or a mixture comprising at least two of said monomers, and/or the
(ii) organic polymer preferably is selected from at least one poly(alkyl-2-acrylic acid alkylester), poly(aryl-2-acrylic acid alkylester), poly(arylalkyl-2-acrylic acid alkylester), each independently having 1 to 20 C-atoms in the alkyl group, each independently having 6 to 14 C-atoms in the aryl group, each independently having 6 to 14 C-atoms in the arylalkyl group, and each independently having 1 to 10 C-atoms in the alkylester group, or a mixture comprising at least two of said polymers.

A bone cement according to the invention can comprise, aside from the soluble organic polymer, in particular polymethylmethacrylate (PMMA), and the monomer for radical polymerisation, in particular methacrylic acid methylester, a particulate inorganic additive, preferably at a concentration of 0.01 to 0.5% by weight, in particular of 0.01 to 0.25% by weight, preferably of 0.02 to 0.14% by weight relative to the total composition. According to the invention, the bone cement dough produced by mixing the powder component and the liquid monomer component comprises the particulate inorganic additive at a concentration of 0.02 to 0.14% by weight. In addition to the components mentioned above, a bone cement according to the invention comprises a radiopaquer, a polymerisation initiator and/or a polymerisation accelerator and, optionally, additional filling agents other than the additive that simply possess a thickening effect.

The particulate inorganic additive can be selected from the group of pyrogenic silicon dioxide, pyrogenic mixed metal-silicon oxides, bentonite, montmorillonite, and a mixture containing at least two of said additives. Moreover, it is also feasible to use pyrogenic silicon dioxide made hydrophobic.

The bone cement, pastes, liquid and/or powder components according to the invention can contain at least one polymerisation initiator (which preferably is soluble in the monomer for radical polymerisation), at least one polymerisation accelerator (which preferably is soluble in the monomer for radical polymerisation), at least one polymerisation co-accelerator, if applicable, or at least one polymerisation initiator, at least one polymerisation accelerator, and, if applicable, at least one polymerisation co-accelerator.

Conceivable as polymerisation initiator are, in particular, peroxides and barbituric acid derivatives, whereby preferably at least 1 g/l, more preferably at least 3 g/l, even more preferably at least 5 g/l, and particularly preferably at least 10 g/l of the peroxides and barbituric acid derivatives can dissolve in the polymerisable monomer at a temperature of 25° C.

According to the invention, a peroxide is understood to mean compounds that contain at least one peroxo group (—O—O—). The peroxide preferably comprises no free acid groups. The peroxide can be an inorganic peroxide or an organic peroxide, such as, for example, a toxicologically acceptable hydroperoxide. According to a particularly preferred embodiment, the peroxide is selected from the group consisting of cumene-hydroperoxide, 1,1,3,3-tetramethylbutyl-hydroperoxide, t-butyl-hydroperoxide, t-amyl-hydroperoxide, di-isopropylbenzen-mono-hydroperoxide, and a mixture of at least two thereof. The barbituric acid derivative preferably is a barbituric acid derivative selected from the group consisting of 1-mono-substituted barbiturates, 5-mono-substituted barbiturates, 1,5-di-substituted barbiturates, and 1,3,5-tri-substituted barbiturates. According to a particular refinement of the paste according to the invention, the barbituric acid derivative is selected from the group consisting of 1,5-di-substituted barbiturates and 1,3,5-tri-substituted barbiturates. It is preferred to use 1,5-disubstituted thiobarbiturates or 1,3,5-trisubstituted thiobarbiturates. According to a preferred embodiment, the substituents each have a length of 1 to 10 carbon atoms. According to a particularly preferred embodiment, the barbituric acid derivative is selected from the group consisting of 1-cyclohexyl-5-ethyl-barbituric acid, 1-phenyl-5-ethyl-barbituric acid, and 1,3,5-trimethyl-barbituric acid.

Polymerisation initiator and polymerisation accelerator can be present each independently in component A and/or B at 0.01 to 5% by weight.

Heavy metal compounds selected from the group consisting of heavy metal salts and heavy metal complexes are preferred as polymerisation accelerator. Heavy metal compounds that are preferred according to the invention are selected from the group consisting of copper(II) hydroxide, copper(II) methacrylate, copper(II) acetylacetonate, copper(II)-2-ethyl-hexanoate, cobalt(II) hydroxide, cobalt(II)-2-ethyl-hexanoate, basic copper(II) carbonate, iron(II)-2-ethyl-hexanoate, iron(III)-2-ethyl-hexanoate, and a mixture of at least two thereof.

According to another embodiment, the bone cement or at least one paste, liquid or powder component can comprise a polymerisation accelerator that is selected from the group consisting of N,N-dimethyl-p-toluidine, N,N-bis-hydroxyethyl-p-toluidine, N,N-dimethyl-aniline, trioctylmethylammoniumchloride, tetrabutylammoniumchloride, lithium chloride, saccharin, 1,8-diazabicyclo[5.4.0]undec-7-ene, and 1,5-diazabicyclo(4.3.0)non-5-ene, phthalimide, maleimide, succinimide, pyromellitic acid diimide, and a mixture of at least two thereof.

Another advantageous refinement of the invention comprises the use, as polymerisation accelerator, of combinations of heavy metal salts and at least one member of the group comprising N,N-dimethyl-p-toluidine, N,N-bis-hydroxyethyl-p-toluidine, N, N-dimethyl-aniline, trioctylmethylammoniumchloride, tetrabutylammoniumchloride, lithium chloride, saccharin, 1,8-diazabicyclo[5.4.0]undec-7-ene, and 1,5-diazabicyclo(4.3.0)non-5-ene, phthalimide, maleimide, succinimide, and pyromellitic acid diimide. In this context, combinations of two and combinations of three different polymerisation accelerators are disclosed in the scope of the invention.

An advantageous refinement of the invention is that the composition according to the invention or any of the pastes A, B or liquid B or powder component A contains at least one co-polymerisation accelerator, if applicable, whereby tertiary amines and amidines are preferred as polymerisation co-accelerators, and whereby N,N-dimethyl-p-toluidine, N,N-bis-hydroxyethyl-p-toluidine, N,N-dimethyl-aniline, 1,8-diazabicyclo[5.4.0-]undec-7-ene, and 1,5-diazabicyclo(4.3.0)-non-5-ene are particularly preferred as co-accelerators/accelerators.

The bone cement according to the invention, in particular in the form of a paste, can contain a (total) amount of the polymerisation initiator, polymerisation accelerator, polymerisation co-accelerator or the polymerisation initiator, polymerisation accelerator, and polymerisation co-accelerator of up to 10% by weight, preferably 1 to 5% by weight, relative to the total weight of the bone cement or, each independent of each other, relative to the total weight of any of the pastes A, B liquid B or powder component A.

The bone cement according to the invention, in particular in the form of a paste, or pastes A, B or liquid B or powder component A, can contain further ingredients aside from the components mentioned above.

The mixing ratio of component A and component B usually is 1:10 to 10:1% by volume, preferably 1:2 to 2:1% by volume.

The radiopaquer can be a common radiopaquer in this field. Suitable radiopaquers can be soluble or insoluble in the monomer for radical polymerisation. The radiopaquer is preferably selected from the group consisting of metal oxides (such as, for example, zirconium dioxide), barium sulfate, toxicologically acceptable heavy metal particles (such as, for example, tantalum), ferrite, magnetite (supramagnetic magnetite also, if applicable), and biocompatible calcium salts. Said radiopaquers preferably have a mean particle diameter in the range of 10 nm to 500 μm. Moreover, conceivable radiopaquers also include esters of 3,5-bis(acetamido)-2,4,6-triiodobenzoic acid, gadolinium compounds, such as gadolinium chelate involving the esters of 1,4,7,10-tetraazacyclododecan-1,4,7,10-tetraacetic acid (DOTA). The radiopaquer concentrations, in particular the zirconium dioxide concentration, in the bone cement or any of the pastes A, B, liquid B or powder component A can, each independent of each other, be in the range of, for example, 3 to 30% by weight relative to the corresponding total composition. Radiopaquers are not considered to be filling agents herein.

The polymerisation stabiliser should be suitable to prevent spontaneous polymerisation of the monomers for radical polymerisation contained in the paste. Moreover, the stabiliser should not undergo interfering interactions with the other ingredients contained in the paste according to the invention. Stabilisers of said type are known according to the prior art. According to a preferred embodiment, the stabiliser is 2,6-di-tert-butyl-4-methylphenol and/or 2,6-di-tert-butyl-phenol.

The antibiotic polymethylmethacrylate bone cement according to the invention is used as revision cement, for one-stage and two-stage septic revision surgeries, for producing spacers and for the manufacture of local release agent carriers. The local release agent carriers can take any suitable three-dimensional shape, such as spherical, bean-shaped or rod-shaped. Shapes that can be extruded or can be obtained in a granulation or compacting method a particularly preferred.

The invention is illustrated through the examples presented in the following, though without limiting the scope of the invention to said examples.

### EXAMPLES

Firstly, a basic cement powder having the following composition was produced by mixing the components in a 1.5 I plastic bottle using a Turbula mixer:

88.9% by weight polymethylmethacrylate-co-methylacrylate (Mw>400,000 g/ml), 10.0% by weight of zirconium dioxide, 1.1% by weight dibenzoylperoxide.

The cement powder survey examples 1-4 were produced by mixing the basic cement powder with daptomycin (activity coefficient AC=943), gentamicin sulfate (Fukang Fujian, activity coefficient AC=580), calcium gluconate (Sigma-Aldrich), and calcium sulfate-dihydrate (Sigma-Aldrich) using a Turbula mixer.

Composition of the cement powders of examples 1-4

| | Composition of the cement powders [g] | | | | | |
|---|---|---|---|---|---|---|
| | Basic | | | | Calcium | Total mass of |
| | cement | | Gentamicin | Calcium | sulfate | the cement |
| Example | powder | Daptomycin | sulfate | gluconate | dihydrate | powder [g] |
| 1 | 40.0 | 0.50 | 0.86 | 0.50 | 0.50 | 42.36 |
| 2 | 40.0 | 0.75 | 0.86 | 0.50 | 0.50 | 42.61 |
| 3 | 40.0 | 1.00 | 0.86 | 0.50 | 0.50 | 42.86 |
| 4 | 40.0 | 1.50 | 0.86 | 0.50 | 0.50 | 43.36 |

Gentamicin sulfate with an activity coefficient of AC = 580
Daptomycin with an activity coefficient of AC = 943

For the subsequent production of test bodies, the cement powder of examples 1 to 4 was mixed with 20 ml of monomer liquid each. The monomer liquid in each case was composed from 18.50 methylmethacrylate, 0.38 g N,N-dimethyl-p-toluidine, 0.002 g hydroquinone, and traces of chlorophyllin (E241). After mixing the cement powders of examples 1-4 with 20 ml of monomer liquid each, a plastically deformable, greenish cement dough was produced after approx. 60 seconds and was used to produce test bodies. The cement dough was cured after approximately 4 minutes.

Strip-shaped test bodies sized 3.3 mm×10.0 mm×75.0 mm were produced for the determination of flexural strength and flexural modulus in accordance with ISO 5833-E/F:2002. Cylinder-shaped test bodies with a diameter of 6.0 mm and a height of 10.0 mm were produced for the determination of the compressive strength. A Zwick Z010 universal testing apparatus was used in the determination of the flexural strength, flexural modulus, and compressive strength in accordance with ISO 5833.

| | Flexural strength | Flexural modulus | Compressive strength |
|---|---|---|---|
| Example | [MPa] | [MPa] | [MPa] |
| 1 | 69.9 ± 1.6 | 3120 ± 95 | 91.8 ± 1.3 |
| 2 | 71.9 ± 1.6 | 3251 ± 60 | 93.4 ± 2.6 |
| 3 | 72.3 ± 1.4 | 3342 ± 113 | 90.3 ± 1.9 |
| 4 | 66.0 ± 2.3 | 3148 ± 168 | 93.3 ± 2.8 |

ISO 5833 requires a flexural strength in excess of 50 MPa, a flexural modulus in excess of 1,800 MPa, and a compressive strength in excess of 70 MPa. The cements of examples 1 to 4 meet the requirements of ISO 5833 with regard to the flexural strength, flexural modulus, and compressive strength.

To test the in-vitro release of daptomycin, gentamicin, and calcium, the cement powders of examples 1 to 4 were used to produce cylindrical test bodies (diameter 25 mm, height 10 mm). For this purpose, one test body each was stored in 20 ml of an aqueous 0.1 M Tris-HCl buffer pH 7.4 at 37° C. for a period of 5 days. Three test bodies of each cement were eluted in parallel. Each day, the elution medium was removed completely and replaced by new elution medium. The daptomycin and gentamicin content of the eluates was determined by HPLC-MS/MS (AZB Biopharm GmbH, Berlin, AZB Report: AZB15-025). The means of the measuring results are shown in the tables below.

| | Gentamicin [μg/test body] | | | |
|---|---|---|---|---|
| Time [d] | Example 1 | Example 2 | Example 3 | Example 4 |
| 1 | 446 ± 134 | 536 ± 72 | 655 ± 103 | 734 ± 48 |
| 2 | 169 ± 17 | 184 ± 25 | 192 ± 38 | 327 ± 58 |
| 3 | 105 ± 23 | 130 ± 27 | 138 ± 13 | 346 ± 138 |
| 4 | 76 ± 11 | 80 ± 14 | 94 ± 4 | 177 ± 68 |
| 5 | 56 ± 10 | 80 ± 14 | 89 ± 20 | 107 ± 8 |

| | Daptomycin [μg/test body] | | | |
|---|---|---|---|---|
| Time [d] | Example 1 | Example 2 | Example 3 | Example 4 |
| 1 | 264 ± 28 | 447 ± 22 | 611 ± 27 | 1040 ± 31 |
| 2 | 18 ± 3 | 27 ± 3 | 42 ± 6 | 95 ± 8 |
| 3 | 10 ± 1 | 16 ± 3 | 24 ± 5 | 43 ± 8 |
| 4 | 9 ± 2 | 14 ± 4 | 19 ± 4 | 43 ± 5 |
| 5 | 6 ± 1 | 9 ± 1 | 16 ± 3 | 28 ± 2 |

ICP-MS was used in the determination of the calcium concentration of the eluates.

| | Calcium [μg/test body] | | | |
|---|---|---|---|---|
| Time [d] | Example 1 | Example 2 | Example 3 | Example 4 |
| 1 | 86 ± 6 | 97 ± 2 | 102 ± 2 | 122 ± 1 |
| 2 | 12 ± 1 | 13 ± 0 | 15 ± 1 | 21 ± 1 |
| 3 | 7 ± 1 | 8 ± 0 | 9 ± 1 | 13 ± 0 |
| 4 | 5 ± 0 | 7 ± 1 | 7 ± 1 | 10 ± 1 |
| 5 | 4 ± 0 | 5 ± 0 | 6 ± 0 | 8 ± 0 |

Moreover, to test the antimicrobial efficacy, cylindrical test bodies (diameter 6 mm, height 15 mm) were produced using the cement powders of examples 1 to 4 and the monomer liquid. These test bodies were used in a CERTIKA proliferation test using Staphylococcus aureus CCUG 45315 (VRSA) as the test germ. The test bodies from the cements of examples 1 to 4 showed complete inhibition of the test germ in the CERTIKA proliferation test (Quality Labs GmbH Nürnberg, Report of Work Order 1935, Measurement 20140925-R06-01-10).

1. A method of conducting a one-stage or two-stage septic revision surgery, said method comprising anchoring a revision articular endoprosthesis in an area within the human body with an antibiotic bone cement, wherein the antibiotic bone cement is prepared by polymerizing an antibiotic polymerizable bone cement comprising:
(i) at least one monomer for radical polymerisation;
(a2) at least one organic polymer comprising at least one polymethylmethacrylate and/or one polymethylmethacrylate-copolymer, and
(iii) at least one polymerisation initiator;
(iv) at least one radiopaquer;
wherein
the antibiotic polymerizable bone cement comprises,
(v) as component 1, daptomycin, a pharmacologically tolerable salt of daptomycin, a polymorphous form of daptomycin, a solvate and/or a hydrate containing daptomycin; and
(vi) at least one calcium salt.

2. Method according to claim 1, wherein the polymerizable bone cement comprises, as
(vi) a combination comprising:
a) as component 2, a water-soluble calcium salt possessing a solubility in water at room temperature of more than or equal to 5 g/l; and
b) as component 3, a poorly water-soluble calcium salt possessing a solubility in water at room temperature of less than 5 g/l; and, optionally,
c) at least one of components 1, 2 and/or 3 has a mean particle size of 1 to 250 μm.

3. Method according to claim 1, which comprises two components A and B, whereby
(i) component A is present as a paste and comprises
(a1) at least one monomer for radical polymerisation;
(a2) at least one organic polymer comprising at least one polymethylmethacrylate and/or one polymethylmethacrylate-copolymer; and
(a3) at least one polymerisation initiator; and
component B is present as a paste and comprises
(b1) at least one monomer for radical polymerisation;
(b2) at least one organic polymer comprising at least one polymethylmethacrylate and/or one polymethylmethacrylate-copolymer; and
(b3) at least one polymerisation accelerator; or
(ii)component A is present as a powder and comprises
(a1) at least one powdered polymer comprising at least one polymethylmethacrylate and/or a powdered mixture comprising polymethylmethacrylate-co-polymers;
(a2) at least one powdered radiopaquer; and
(a3) at least one polymerisation initiator; and
component B is present as a liquid or paste and comprises
(b1) at least one monomer for radical polymerisation;
(b2) optionally, at least one organic polymer comprising at least one polymethylmethacrylate and/or one polymethylmethacrylate-copolymer; and
(b3) at least one polymerisation accelerator; and
whereby at least component A comprises, as
(a4), at least one of components 1, 2, and 3 selected from component 1 daptomycin, a pharmacologically tolerable salt of daptomycin, a solvate and/or a hydrate containing daptomycin, component 2 a water-soluble calcium salt possessing a solubility in water at room temperature of more than or equal to 5 g/l and component 3 a poorly water-soluble calcium salt possessing a solubility in water at room temperature of less than 5 g/l, and/or
whereby component B is present as a paste and comprises, as
(b4), at least one of components 1, 2, and 3 selected from component 1 daptomycin, a pharmacologically tolerable salt of daptomycin, a polymorphous form of daptomycin, a solvate and/or a hydrate containing daptomycin, component 2 a water-soluble calcium salt possessing a solubility in water at room temperature of more than or equal to 5 g/l, and component 3 a poorly water-soluble calcium salt possessing a solubility in water at room temperature of less than 5 g/l.

4. Method according to claim 1, wherein
(v) component 1 daptomycin, a pharmacologically tolerable salt of daptomycin, a polymorphous form of daptomycin, a solvate and/or a hydrate containing daptomycin, and (vi) a) component 2 the water-soluble calcium salt possessing a solubility in water at room temperature of more than or equal to 5 g/l, and b) component 3 the poorly water-soluble calcium salt possessing a solubility in water at room temperature of less than 5 g/l, each independently is present in the form of particles of components 1, 2 or 3 or each independently is a particulate formulation containing, each independently, at least one of components 1, 2 and/or 3, and at least one pharmacological excipient and, optionally, each independently comprise a mean particle size of 1 to 250 μm.

5. Method according to claim 1, wherein at least component A comprises, as
(a4), at least one of components 1, 2, and 3, which each independently are present in the form of particles of components 1, 2 or 3 or each independently, as a particulate formulation, containing, each independently, at least one of components 1, 2 and/or 3 and, optionally, each independently comprise a mean particle size of 1 to 250 μm,
whereby component B is present as a paste and comprises, as (b4) at least one of components 1, 2, and 3, which each independently are present in the form of particles of components 1, 2 or 3 or each independently, as a particulate formulation, containing, each independently, at least one of components 1, 2 and/or 3 and, optionally, each independently comprise a mean particle size of 1 to 250 μm.

6. Method according to claim 1, wherein
a) the polymerizable bone cement is present, as (a4) and/or (b4), as particles of a combination at least of components 1 and 2 or, each independently, as a particulate formulation containing at least a combination of components 1 and 2 and at least one pharmacological excipient; or
b) the polymerizable bone cement is present, as (a4) and/or (b4), as particles of a combination of components 1, 2, and 3 or, each independently, as a particulate formulation containing the combination of components 1, 2, and 3 and at least one pharmacological excipient.

7. Method according to claim 2, wherein
a) component 2, the water-soluble calcium salt possessing a solubility in water at room temperature of more than or equal to 5 g/l, comprises calcium gluconate, calcium glucuronate, calcium lactate, calcium acetate and/or calcium sorbate or a mixture containing at least two of said calcium salts.

8. Method according to claim 2, wherein
b) component 3 the poorly water-soluble calcium salt possessing a solubility in water at room temperature of less than 5 g/l, comprises calciumsulfate-dihydrate, calciumsulfat-hemihydrate, alpha-tricalciumphosphate and/or beta-tricalciumphosphate.

9. Method according to claim 1, which contains at least one second antibiotic selected from the group of the aminoglycoside antibiotics and/or the lincosamide antibiotics and/or the ansamycin antibiotics and/or the fluoroquinolone antibiotics and/or the β-lactam antibiotics.

10. The method according to claim 1, wherein the area within the human body has been subjected to radical debridement after explantation of an infected articular endoprosthesis.

11. The method according to claim 10, wherein the infected articular endoprosthesis is infected with gram-positive bacteria.

12. The method according to claim 11, wherein the gram-positive bacteria is selected from the group consisting of methicillin-resistant staphylococci and vancomycin-resistant staphylococci.

13. The method according to claim 12, wherein the gram-positive bacteria is selected from the group consisting of vancomycin-resistant staphylococci.

14. The method according to claim 1, wherein the antibiotic bone cement is adapted to provide synchronous release of daptomycin and calcium ions over a period of several days.

15. The method according to claim 1, wherein the antibiotic bone cement is in the form of a revision implant, screw, nail, or surgical plate used to mechanically fix the revision articular endoprosthesis in place.

16. A method of conducting a one-stage or two-stage septic revision surgery, said method comprising removing an infected articular endoprosthesis from an area within the human body and temporarily introducing a spacer into that area, wherein the spacer is composed of an antibiotic bone cement prepared by polymerizing an antibiotic polymerizable bone cement comprising:
(i) at least one monomer for radical polymerisation;
(a2) at least one organic polymer comprising at least one polymethylmethacrylate and/or one polymethylmethacrylate-copolymer; and
(iii) at least one polymerisation initiator;
(iv) at least one radiopaquer;
wherein
the antibiotic polymerizable bone cement comprises,
(v) as component 1, daptomycin, a pharmacologically tolerable salt of daptomycin, a polymorphous form of daptomycin, a solvate and/or a hydrate containing daptomycin; and
(vi) at least one calcium salt.

17. Method according to claim 16, wherein the polymerizable bone cement comprises, as
(vi) a combination comprising:
a) as component 2, a water-soluble calcium salt possessing a solubility in water at room temperature of more than or equal to 5 g/l; and
b) as component 3, a poorly water-soluble calcium salt possessing a solubility in water at room temperature of less than 5 g/l; and, optionally,
c) at least one of components 1, 2 and/or 3 has a mean particle size of 1 to 250 μm.

18. Method according to claim 16, which comprises two components A and B, whereby
(i) component A is present as a paste and comprises
(a1) at least one monomer for radical polymerisation;
(a2) at least one organic polymer comprising at least one polymethylmethacrylate and/or one polymethylmethacrylate-copolymer; and
(a3) at least one polymerisation initiator; and component B is present as a paste and comprises
(b1) at least one monomer for radical polymerisation;
(b2) at least one organic polymer comprising at least one polymethylmethacrylate and/or one polymethylmethacrylate-copolymer; and
(b3) at least one polymerisation accelerator; or
(ii)component A is present as a powder and comprises
(a1) at least one powdered polymer comprising at least one polymethylmethacrylate and/or a powdered mixture comprising polymethylmethacrylate-co-polymers;
(a2) at least one powdered radiopaquer; and
(a3) at least one polymerisation initiator; and
component B is present as a liquid or paste and comprises
(b1) at least one monomer for radical polymerisation;
(b2) optionally, at least one organic polymer comprising at least one polymethylmethacrylate and/or one polymethylmethacrylate-copolymer; and
(b3) at least one polymerisation accelerator; and
whereby at least component A comprises, as
(a4), at least one of components 1, 2, and 3 selected from component 1 daptomycin, a pharmacologically tolerable salt of daptomycin, a solvate and/or a hydrate containing daptomycin, component 2 a water-soluble calcium salt possessing a solubility in water at room temperature of more than or equal to 5 g/l and component 3 a poorly water-soluble calcium salt possessing a solubility in water at room temperature of less than 5 g/l, and/or whereby component B is present as a paste and comprises, as
(b4), at least one of components 1, 2, and 3 selected from component 1 daptomycin, a pharmacologically tolerable salt of daptomycin, a polymorphous form of daptomycin, a solvate and/or a hydrate containing daptomycin, component 2 a water-soluble calcium salt possessing a solubility in water at room temperature of more than or equal to 5 g/l, and component 3 a poorly water-soluble calcium salt possessing a solubility in water at room temperature of less than 5 g/l.

19. Method according to claim 16, wherein
(v) component 1 daptomycin, a pharmacologically tolerable salt of daptomycin, a polymorphous form of daptomycin, a solvate and/or a hydrate containing daptomycin, and (vi) a) component 2 the water-soluble calcium salt possessing a solubility in water at room temperature of more than or equal to 5 g/l, and b) component 3 the poorly water-soluble calcium salt possessing a solubility in water at room temperature of less than 5 g/l, each independently is present in the form of particles of components 1, 2 or 3 or each independently is a particulate formulation containing, each independently, at least one of components 1, 2 and/or 3, and at least one pharmacological excipient and, optionally, each independently comprise a mean particle size of 1 to 250 μm.

20. Method according to claim 16, wherein at least component A comprises, as
(a4), at least one of components 1, 2, and 3, which each independently are present in the form of particles of components 1, 2 or 3 or each independently, as a particulate formulation, containing, each independently, at least one of components 1, 2 and/or 3 and, optionally, each independently comprise a mean particle size of 1 to 250 μm,
whereby component B is present as a paste and comprises, as (b4) at least one of components 1, 2, and 3, which each independently are present in the form of particles of components 1, 2 or 3 or each independently, as a particulate formulation, containing, each independently, at least one of components 1, 2 and/or 3 and, optionally, each independently comprise a mean particle size of 1 to 250 μm.

21. Method according to claim 16, wherein
a) the polymerizable bone cement is present, as (a4) and/or (b4), as particles of a combination at least of components 1 and 2 or, each independently, as a particulate formulation containing at least a combination of components 1 and 2 and at least one pharmacological excipient; or
b) the polymerizable bone cement is present, as (a4) and/or (b4), as particles of a combination of components 1, 2, and 3 or, each independently, as a particulate formulation containing the combination of components 1, 2, and 3 and at least one pharmacological excipient.

22. Method according to claim 17, wherein
a) component 2, the water-soluble calcium salt possessing a solubility in water at room temperature of more than or equal to 5 g/l, comprises calcium gluconate, calcium glucuronate, calcium lactate, calcium acetate and/or calcium sorbate or a mixture containing at least two of said calcium salts.

23. Method according to claim 17, wherein
b) component 3 the poorly water-soluble calcium salt possessing a solubility in water at room temperature of less than 5 g/l, comprises calciumsulfate-dihydrate, calciumsulfat-hemihydrate, alpha-tricalciumphosphate and/or beta-tricalciumphosphate.

24. Method according to claim 16, which contains at least one second antibiotic selected from the group of the aminoglycoside antibiotics and/or the lincosamide antibiotics and/or the ansamycin antibiotics and/or the fluoroquinolone antibiotics and/or the β-lactam antibiotics.

25. The method according to claim 16, wherein the area within the human body has been subjected to radical debridement after explantation of an infected articular endoprosthesis.

26. The method according to claim 25, wherein the infected articular endoprosthesis is infected with gram-positive bacteria.

27. The method according to claim 26, wherein the gram-positive bacteria is selected from the group consisting of methicillin-resistant staphylococci and vancomycin-resistant staphylococci.

28. The method according to claim 27, wherein the gram-positive bacteria is selected from the group consisting of vancomycin-resistant staphylococci .

29. The method according to claim 16, wherein the antibiotic bone cement is adapted to provide synchronous release of daptomycin and calcium ions over a period of several days.
